(19) United States
(12) Patent Application Publication
(10) Pub. No.: US 2023/0414962 A1
(43) Pub. Date: Dec. 28, 2023

(54) LIGHT THERAPY SYSTEM

(71) Applicant: Mark Sawyer, Lake Mary, FL (US)

(72) Inventor: Mark Sawyer, Lake Mary, FL (US)

(21) Appl. No.: 17/961,258

(22) Filed: Oct. 6, 2022

Related U.S. Application Data
(60) Provisional application No. 63/354,958, filed on Jun. 23, 2022.

(51) Int. Cl. A61N 5/06

(52) U.S. Cl. CPC A61N 5/06; A61N 2005/0652; A61N 2005/0658; A61N 2005/0627

(57) ABSTRACT

A light therapy system includes a remote-control device and at least one light therapy device. Each light therapy devices include a main body, a plurality of optical emitters, a system controller and an onboard user interface device. The light therapy devices operate in a standalone mode using the onboard interface device and a cumulative mode where operating instructions are received from the remote-control device. The operating instructions are distinct and individual for each light therapy device, to permit identical or different operation between devices. The light therapy devices do not communicate directly with each other and are operated wirelessly by the remote-control device such that a failure in one light therapy device does not affect the operation of another light therapy device.

### CROSS-REFERENCE TO RELATED APPLICATIONS

This application claims the benefit of U.S. Application Ser. No. 63/354,958 filed on Jun. 23, 2022, the contents of which are incorporated herein by reference.

### TECHNICAL FIELD

The present invention relates generally to therapy devices, and more particularly to a light therapy system having at least one remotely operable and independently configurable light therapy device.

### BACKGROUND

The statements in this section merely provide background information related to the present disclosure and may not constitute prior art.

Lighting units for use in agricultural settings have been used for decades to generate photons for use by plants for photosynthesis. More recently, scientific research has shown that exposing humans to certain types of light can have a meaningful effect on a person's overall health. Depending on the intensity and wavelength of the light, these effects can include reducing stress, eliminating headaches, promoting hair growth, increasing collagen production, and even fat reduction.

For these reasons, there are several known types of light therapy devices currently on the market. Such devices typically comprise a panel having an array of individual lights that are controlled by an onboard controller. A switch located directly on the panel is physically activated by a user to transition the device between an ON and OFF operating state.

Although useful, these devices are typically too small to treat large areas of a human user at a single time. As such, it is not uncommon for multiple devices to be connected when treating a user. In order to function optimally, each of the connected devices must operate at the same output levels and must switch between operating modes at the same time. For this reason, such systems rely on a communication protocol wherein each device is physically linked together by a cable in a serial manner such that a first unit controls the operation of a subsequent unit in a “master/slave” or “lead/follow” relationship.

Unfortunately, this arrangement suffers from several practical drawbacks. For example, the requirement to physically connect a series of individual lighting devices together by a communication cable restricts the ability of a user to position multiple devices at different positions when treating a user. Moreover, when multiple devices are interconnected, there is a noticeable lag between the actions of the first panel in the chain and the last panel due to the processing time required by the onboard controller of each device and the speed limitations imposed by the serial communication cables. As such, this lag increases proportionally with the number of additional panels that are connected to the chain.

In addition to the above, there are serious operational issues when a single device in the chain malfunctions. To this end, when multiple devices are used together in the lead/follow orientation, and any device in the chain fails, all devices located downstream from the affected device also fail. When this occurs, the user (who is often wearing protective eyewear) may not be aware of the failure and thus may not receive the treatment they are expecting. Worse, is if one of the panels malfunctions or operates in an output mode that is higher than what was instructed at the first device. When such an issue occurs, the subsequent output of the overall system comprising multiple devices may be well above the recommended or set output at the first device, thus potentially risking harm to the user.

In both instances, a technician must be present to identify the malfunctioning device, physically disconnect that device from the overall system, and then rewire the remaining devices to resume or begin functioning correctly.

Accordingly, it would be beneficial to provide a system having one or more light therapy devices that can each be independently operated by a remote-control device so as to be capable of functioning alone or in combination with other devices without the drawbacks described above.

### SUMMARY OF THE INVENTION

The present invention is directed to a light therapy system. One embodiment of the present invention can include a remote-control device and at least one light therapy device. Each of the light therapy devices can include a main body, a plurality of optical emitters, a system controller, and an onboard user interface device.

In one embodiment, the plurality of light therapy devices can operate in a standalone mode wherein operating instructions are received from the onboard interface device or the remote-control device and implemented by the onboard controller. In one embodiment, the plurality of light therapy devices can also operate in a cumulative mode where operating instructions are received from the remote-control device. The operating instructions can be distinct and individual for each light therapy device, so as to allow each device to operate in the same manner or in a different manner than the other light therapy devices that are connected to the remote-control.

When operating in the cumulative mode, the plurality of light therapy devices do not communicate directly with each other, and can be operated individually and wirelessly by the remote-control device such that a failure in one light therapy device does not affect the operation of another light therapy device.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of a light therapy system that is useful for understanding the inventive concepts disclosed herein.

FIG. 2 is a perspective view of a light therapy device of the light therapy system, in accordance with one embodiment of the invention.

FIG. 3 a simplified block diagram of the system controller of the light therapy device, in accordance with one embodiment of the invention.

FIG. 4 is another perspective view of the light therapy system, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

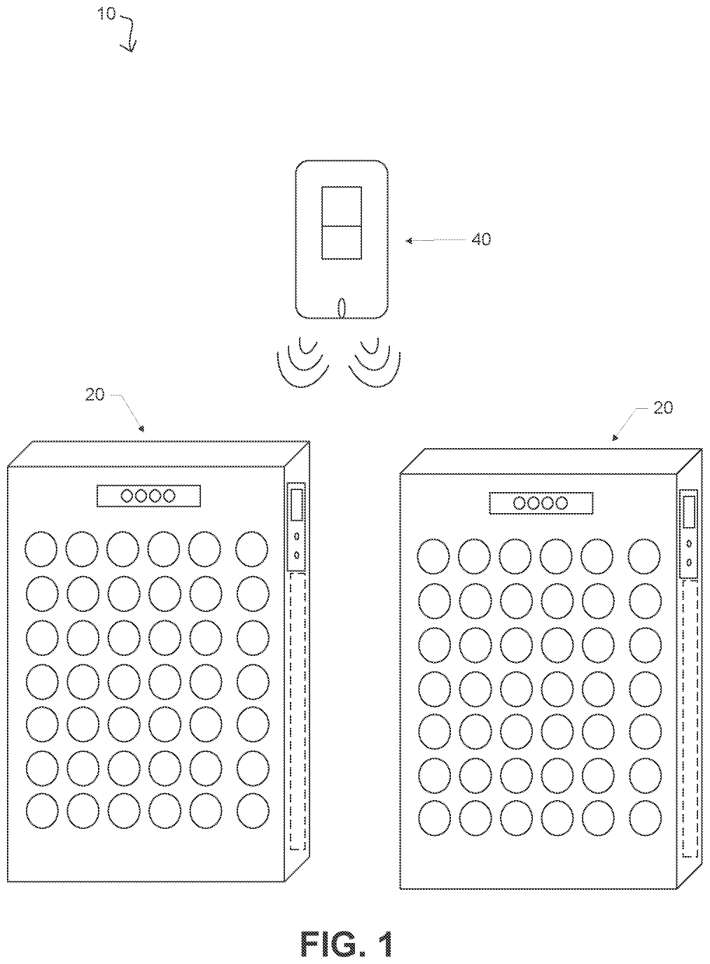

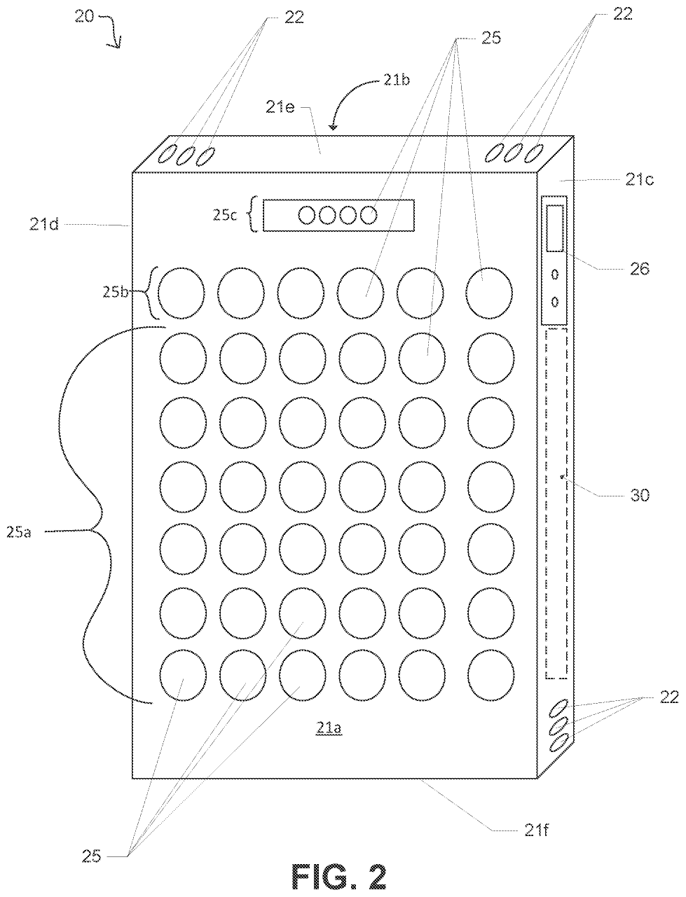

FIGS. 1-4 illustrate one embodiment of a light therapy system 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 2.

As shown and described herein, the light therapy system 10 can include one or more individual light therapy devices 20, that are independently and cumulatively operated by a single remote-control device 40.

As shown best at FIG. 2, each light therapy device 20 can include a main body having a front wall 21a, a back wall 21b, a pair of side walls 21c and 21d, a top wall 21e and a bottom wall 21f that define an interior space. In the preferred embodiment, the main body will be constructed from stamped steel panels and will include a generally rectangular shape having a plurality of air vents 22 along each of the top, bottom, back and side walls.

Of course, other embodiments are contemplated wherein the main body includes any number of different shapes and sizes. Moreover, the main body may be formed from any number of other materials that are, for example, relatively strong and stiff for their weight. Several nonlimiting examples include but are not limited to various metals or metal alloys (e.g., aluminum, titanium, or alloys thereof), plastic/polymers (e.g., high-density polyethylene (HDPE), rigid polyvinyl chloride (PVC), or polyethylene terephthalate (PET)), and/or various composite materials (e.g., carbon fibers in a polymer matrix, fiberglass, etc.).

In one embodiment, a plurality of optical emitters 25 can be positioned along or within the main body so as to direct optical emissions outward therefrom in a specified direction. Each of the optical emitters 25 can be communicatively linked to the below described controller 30 in order to receive operating instructions and/or power therefrom. In the preferred embodiment, each of the optical emitters can comprise light emitting diodes (LED's); however, any number of other types of devices capable of producing optical emissions are also contemplated.

In one embodiment, the plurality of optical emitters can be divided into different groups or arrays such as arrays 25a, 25b and 25c, for example. To this end, each array can produce an optical output/output emission that is the same as another array or that is different from another array. For example, in one nonlimiting embodiment, array 25a can function to produce visible red light at any desirable wavelength suitable for visible red-light therapy, whereas array 25b can function to produce infrared light at any desirable wavelength suitable for infrared therapy.

In the same nonlimiting exemplary embodiment, array 25c can produce visible light in any number of different colors and intensities so as to provide a visual indication to a device user of the operating mode at which the device is operating. The presence of array 25c on the front wall of the device is specifically advantageous, as it allows a user to constantly monitor the operating status of the device without having to look away or move to the side of the device to see the control panel.

Although described above with regard to a specific number of arrays operating at specific outputs, this is for illustrative purposes only. To this end, each device 20 can include any number of different arrays each having any number of different optical emitters capable of producing light in any color and in any of the visible, near-infrared, infrared, far-infrared and/or other spectrums. Moreover, some of the optical emitters can include a different construction than other emitters. Several nonlimiting examples of other types of optical emitters which may be used herein include heat bulbs, incandescent bulbs, LCD, and/or OLED elements, among others, for example. Such a feature allowing a single device to be capable of performing light therapy at multiple different wavelengths and emission parameters at the same time so as to treat different ailments simultaneously.

In one embodiment, a user interface 26 can be communicatively linked to the system controller 30 and can be positioned along one of the walls of the main body. In the preferred embodiment, the user interface device can include a Graphic User Interface (GUI) screen for providing two-way communication with a user. To this end, the interface can allow the user to instruct the system to perform functions such as transitioning between an on and off operating state, selectively activating individual optical emitters, selectively activating specific arrays of optical emitters, and for selecting the type and durational output of the same.

In one embodiment, the user interface can include functionality for allowing the user to designate a plurality of specific optical emitters 25 in order to create one or more user-defined arrays. For example, in one nonlimiting embodiment, the user can create a first array comprising the top 4 rows of emitters, and a second array comprising the bottom 4 rows of emitters. Each of the user-defined arrays being capable of operating in the same manner described above with regard to arrays 25a-25c wherein each array can produce output emissions that are the same as another user-defined array or that are different from another user-defined array.

Of course, any number of other user-defined arrays can be created by the user via the user interface device. Such a feature advantageously providing a fully user customizable light therapy device which can be tailored to the specific and different requirements of a user each time they use the device. Additionally, the user interface device is not limited to the use of a touch screen system, as any number of other types of devices such as non-touch screen displays, buttons, switches and/or other input mechanisms are also contemplated.

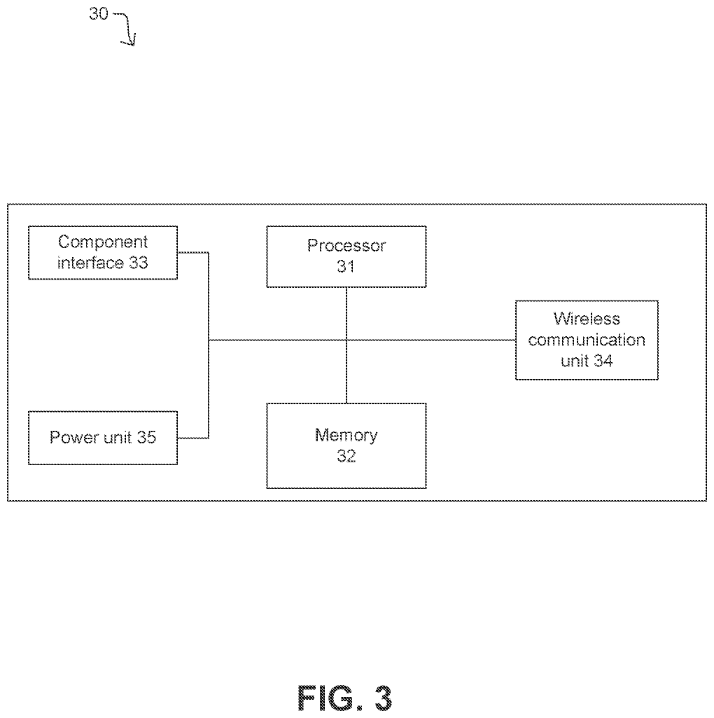

FIG. 3 is a simplistic block diagram illustrating one embodiment of the controller 30 that includes a processing unit 31 that is conventionally connected to an internal memory 32, a component interface unit 33, a wireless communication unit 34, and/or a power unit 35.

Although illustrated as separate elements, those of skill in the art will recognize that one or more system components may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. The bulk storage device can contain any number of different programs that permit the processor to perform the functionality described herein and can also receive and store the exercise information for each user.

The component interface unit 33 can function to provide a communicative link between the processing unit 31, the optical emitters 25 and the user interface device 26. In this regard, the component interface unit can include any number of different components such as one or more PIC microcontrollers, standard bus, internal bus, connection cables, wireless receiver and/or associated hardware such as USB cables and connectors, and other such hardware capable of linking the various components. Of course, any other means for providing the two-way communication between the system components can also be utilized herein.

The communication unit 34 can include any number of components capable of sending and/or receiving electronic signals with another device, either directly or over a network. In the preferred embodiment, the communication unit 34 can include a Wi-Fi transceiver for communicating directly with the remote-control device 40. In another embodiment, the communication unit 34 can include a Wi-Fi or Bluetooth transceiver which can communicate with a user device such as a tablet or smartphone, for example, that is running a mobile application that can be downloaded onto the user device and installed as an application.

Of course, any number of other transmission and reception mechanisms and protocols can also be utilized herein, several nonlimiting examples include cellular transceivers, Near-Field-Communication (NFC) devices, radio, infrared (IR), RFID, and/or network adapter(s) functioning to communicate over a WAN, LAN, or the internet via an internet service provider, among others, for example.

The power unit 35 can include any number of different components capable of providing the necessary power requirements to each element of the device. In the preferred embodiment, the power source can include or comprise an A/C electrical power transformer and cord for allowing the device to be plugged into a standard electrical outlet. Of course, other embodiments are contemplated wherein the power unit includes onboard batteries so as to operate without the need to connect to a power outlet.

In operation, each of the light therapy devices 20 can operate independently via the user interface device so as to function in a standalone mode. Additionally, each of the light therapy devices can operate in a cumulative mode wherein each device is controlled simultaneously by the below described remote-control device.

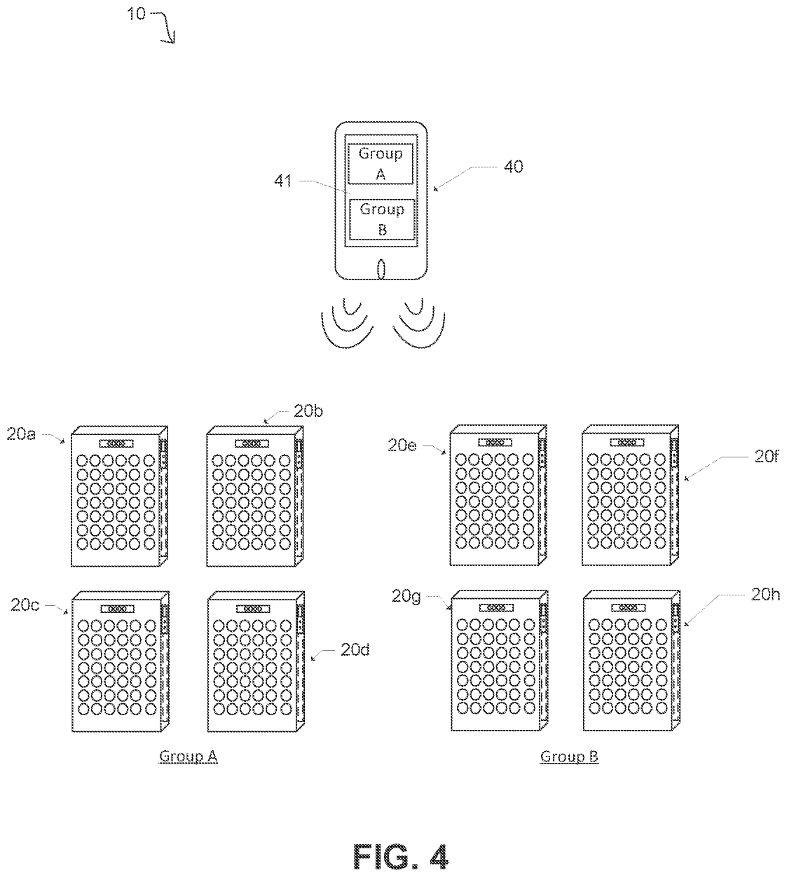

As shown best at FIG. 4, the remote-control device 40 can function to selectively control the operation of any number of individual light therapy devices both individually and as a group. To this end, the remote-control device can include onboard circuitry such as a processor, memory, component interface, wireless interface, and power source substantially identical to that described above with regard to the system controller of the light therapy device.

In one embodiment, the remote-control device can include a small portable housing having a user interface 41 such as individual buttons or a Graphic User Interface (GUI) screen for receiving user instructions and for wirelessly sending operating instructions to one or more of the light therapy devices. Several nonlimiting examples of operating instructions include, but are not limited to a device pairing instruction, device operating mode instructions, device array selection instructions, and/or device array creation instructions, among others for example.

In one embodiment, the system 10 can be configured so that the remote-control device operates in an “on demand” mode. In this configuration, each light therapy device will start, stop, or change a respective operation only upon receipt of an instruction from the remote-control device 40, and will otherwise continue to operate in its previously instructed state until another instruction is received.

In another embodiment, the system can be configured so that the remote-control device operates in a continuous or semi-continuous mode. In this configuration, each light therapy device will automatically transition to an OFF operating state if a signal from the remote-control device is not received within a predetermined timeframe such as every 30 seconds, for example. Such a feature allows the remote-control device to function with other types of light therapy devices that do not have an onboard memory and therefore must receive continuous instructions to continue operating in a desired manner.

By automatically terminating the operation of each light therapy device in the absence of a contrary instruction from the remote-control device, the system operating in the continuous or semi-continuous mode advantageously provides a built-in safety mechanism for ensuring users are not exposed to the therapy device for longer than the recommended timeline.

When used with a plurality of individual therapy devices 20 in the cumulative mode, the remote-control 40 can function to send individualized operating instructions to each device at the same time, in order to allow each device 20 to operate in unison with the other system light therapy devices. Moreover, because each of the light therapy devices are communicatively isolated from each other—e.g., no individual light therapy device is connected to, controls or otherwise communicates with another light therapy device—the system advantageously ensures that a failure by any one light therapy device will have no effect on any of the other light therapy devices in the system.

Additionally, because the light therapy devices are not physically connected together, it is possible to position each device anywhere within a room or other location without being constrained by the positioning of communication cables, as the distances are limited only by the range of the wireless transceiver of the remote-control unit.

In one embodiment, the remote-control can include functionality for allowing a remote-control user to individually select and group different light therapy devices together to form any number of device groupings (e.g., individualized operating instructions). For example, if the remote-control is operating eight light therapy devices 20a, 20b, 20c, 20d, 20e, 20f, 20g and 20h, the user can group several of those into a first group “Group A”, and the remaining devices into a second group “Group B”. In this regard, the remote-control can send different instructions to the different groups based on any number of user criteria.

The ability to send these individualized operating instructions to multiple devices via a single remote is particularly useful in a commercial setting such as a therapist office, for example, wherein multiple light therapy devices are used in different treatment rooms for treating different patients simultaneously. Such a feature allowing a single remote-control device to direct the operation of each group of light therapy devices in each treatment room so as to start, stop or change the operating mode without interfering with the operation of the other groups located in a different therapy room.

Accordingly, the above described system functions in a novel manner by providing a system that allows a user to: 1) configure a plurality of light therapy devices to each operate in a user customized manner via predetermined or user-customized arrays of optical emitters, 2) position each of the light therapy devices in any desirable location, 3) individually control each device via a single remote-control device such that a failure in one device does not affect the operation of another device, and 4) separate the plurality of user-customized devices into user customized groups.

As to a further description of the manner and use of the present invention, the same should be apparent from the above description. Accordingly, no further discussion relating to the manner of usage and operation will be provided.

As described herein, one or more elements of each of the light therapy devices can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A light therapy system, comprising:
a remote-control device;
a first light therapy device having a main body, a plurality of optical emitters, a first system controller and a first user interface device,
said first light therapy device including functionality for receiving operating instructions from the first user interface device to operate in a standalone mode, and for receiving operating instructions from the remote-control device to operate in a cumulative mode; and
a second light therapy device having a main body, a plurality of optical emitters, a second system controller and a second user interface device,
said second light therapy device including functionality for receiving operating instructions from the second user interface device to operate in a standalone mode, and for receiving operating instructions from the remote-control device to operate in a cumulative mode,
wherein the first light therapy device and the second light therapy devices are communicatively isolated from each other, and
wherein the remote-control device includes functionality for communicating wirelessly with each of the first light therapy device and the second light therapy device.

2. The system of claim 1, wherein the remote-control device is configured to send individualized operating instructions to each of the first light therapy device and the second light therapy device.

3. The system of claim 1, wherein when operating in a cumulative mode, an instruction input by the first user interface device will affect the operation of the first light therapy device and will not affect the operation of the second light therapy device.

4. The system of claim 1, wherein when operating in a cumulative mode, an instruction input by the second user interface device will affect the operation of the second light therapy device and will not affect the operation of the first light therapy device.

5. The system of claim 1, wherein when operating in a cumulative mode, an operating instruction received from the remote-control device will override an input by the first user interface device affecting the operation of the first light therapy device.

6. The system of claim 1, wherein when operating in a cumulative mode, an operating instruction received from the remote-control device will override an input by the second user interface device affecting the operation of the second light therapy device.

7. The system of claim 1, wherein the plurality of optical emitters of the first light device comprise light emitting diodes.

8. The system of claim 7, wherein the plurality of optical emitters of the first light therapy device are configured to output light in at least one of a visible, near-infrared, infrared, or far-infrared spectrum.

9. The system of claim 8, wherein the plurality of optical emitters of the first light therapy device are configured to simultaneously output light in at least two of the visible, near-infrared, infrared, or far-infrared spectrums.

10. The system of claim 1, wherein the plurality of optical emitters of the second light device comprise light emitting diodes.

11. The system of claim 10, wherein the plurality of optical emitters of the second light therapy device are configured to output light in at least one of a visible, near-infrared, infrared, or far-infrared spectrum.

12. The system of claim 11, wherein the plurality of optical emitters of the second light therapy device are configured to simultaneously output light in at least two of the visible, near-infrared, infrared, or far-infrared spectrums.
